(19) United States
(12) Patent Application Publication
(10) Pub. No.: US 2022/0257807 A1
(43) Pub. Date: Aug. 18, 2022

(54) STERILIZATION UNIT AND METHODS OF USING THE SAME

(71) Applicant: SANFILIPPO TECH, LLC., Schaumburg, IL (US)

(72) Inventors: John Sanfilippo, Schaumburg, IL (US); James J. Sanfilippo, Schaumburg, IL (US)

(21) Appl. No.: 17/670,220

(22) Filed: Feb. 11, 2022

Related U.S. Application Data
(60) Provisional application No. 63/200,096, filed on Feb. 12, 2021.

(51) Int. Cl. A61L 2/00; A61L 2/26; A61L 2/20; B65B 1/08; B65B 55/18; B65B 55/02

(52) U.S. Cl. CPC A61L 2/0094; A61L 2/26; A61L 2/202; A61L 2/208; A61L 2/206; B65B 1/08; A61L 2202/121; B65B 55/027; A61L 2202/21; A61L 2202/14; A61L 2202/18; A61L 2202/122; B65B 55/18

(57) ABSTRACT

A sterilization unit for sterilizing a product can include a body having an internal volume for receiving the product to be sterilized, and comprising at least one opening through which product can be introduced and/or withdrawn from the internal volume; a gas inlet in fluid communication with the internal volume for introducing one or more of an inert gas and a sterilizing gas into the internal volume during sterilization; a screen disposed in the gas inlet through which the one or more of the inert gas and the sterilizing gas must pass before entering into the internal volume; and a pressure release valve. The body can be capable of being pressurized to a pressure of 10 atm or more.

### CROSS-REFERENCE TO RELATED APPLICATION

This application claims the benefit of priority to U.S. Provisional Patent Application No. 63/200,096 filed Feb. 12, 2021, the disclosure of which is incorporated herein by reference in its entirety.

### BACKGROUND

### Field of the Disclosure

The disclosure relates to a sterilizer unit and method of using the same, and more particularly to a sterilizer unit for sterilizing cannabis and/or hemp products and methods of sterilizing cannabis and/or hemp products using the same.

### Brief Description of Related Technology

Product sterilization is important and widely used in a variety of industries, such as the food and pharmaceutical industries. Various methods of sterilization have been developed depending on the product to be sterilized. Recent regulations have made sterilization a focus in the cannabis industry. Cannabis and hemp products are flowable products and can be particularly sensitive to external stimulus, resulting in discoloration or other damage to the product. Damage can include adverse effects on the flavoring of the product, such as destruction or modification of terpenes in or added to cannabis products, and/or reduction of the effectiveness, such as changes in the THC levels. Cannabis, by its nature as a plant product can also have inherent levels of contaminants that result in the environment in which the cannabis is processed having high airborne levels of contaminants making it difficult to maintain sterility of the product even after effective sterilization. Contaminants can include mold spores, bacteria and viruses.

Some conventional techniques for sterilizing cannabis products include vacuum sterilization relying upon the generation of sub-atmospheric pressures within a chamber that includes a gaseous or aerosolized sterilization reagent. One such system is described in International Patent Application Publication No. WO 2019/133952. Such systems require that the product be contained within a sterilization package, such as a Tyvek pouch to protect the product from the sterilization environment, including the sterilizing reagents present in the chamber. The generation of vacuum can require significant time and energy costs, as well, requiring longer sterilization times to achieve effectiveness. Longer sterilization and prolonged exposure of the product to the sterilization reagents can increase the potential for damage to the product.

### SUMMARY

Embodiments of the sterilization unit and methods of the disclosure advantageously utilize positive pressure differentials combined with flushes of inert and sterilizing gases over the product to more effectively sterilize the product. Units and methods of the disclosure can be particularly advantageous for sterilization of products such as cannabis and hemp because it can allow for the sterilization of loose product without the need to prepackage the product within a sterilization pouch, while maintaining gentle and substantially damage free sterilization of the product. The presence of the product in loose form can also allow for more effective sterilization in the units and methods of the disclosure, which can achieve penetration of the sterilizing gas into interstitial spaces in the product, as well as effective removal of the sterilizing gas from the interstitial space after sterilization to prevent unnecessary prolonged contact of the product with sterilizing gas which could cause damage.

In embodiments a sterilization unit for sterilizing a product can include a body having an internal volume for receiving the product to be sterilized, and comprising at least one opening adapted to be sealed through which product can be introduced and/or withdrawn from the internal volume; a gas inlet in fluid communication with the internal volume for introducing one or more of an inert gas and a sterilizing gas into the internal volume during sterilization; a screen disposed in the gas inlet through which the one or more of the inert gas and the sterilizing gas must pass before entering into the internal volume; and a pressure release valve. The body can be capable of being pressurized to a pressure of 10 atm or more. In embodiments, the unit can include a door for sealing over the opening during pressurization.

In embodiments a sterilization unit for sterilizing a product can include a body having an internal volume for receiving the product to be sterilized and comprising a product inlet end having a product inlet through which product can be introduced into the internal volume and an oppositely disposed product outlet end having a product outlet through which product can be removed from the internal volume through gravitational flow; a product inlet door configured to shift between a first position in which the product inlet door seals the body at the product inlet end and a second position in which the product inlet door is disposed away from the product inlet end and exposes the product inlet to allow product to be introduced into the internal volume; a product outlet door configured shift between a first position in which the product inlet door seals the body at the product outlet end and a second position in which the product outlet door is disposed away from the product outlet end to expose the product outlet and allow product to flow out from the internal volume; a gas inlet in fluid communication with the internal volume for introducing one or more of air and a sterilizing gas into the body during sterilization; a screen disposed in the gas inlet through which the one or more of the air and sterilizing gas much pass before entering into the body; and a pressure release valve. The body can be capable of being pressurized to a pressure of at least 10 atm.

In embodiments, a method of sterilizing a cannabis or hemp product, can include introducing the product into an internal volume of a body of a sterilizer unit and sealing the internal volume, wherein the sterilizer unit further comprises at least one gas inlet in fluid communication with the internal volume for pressurizing the body and at least one release valve for releasing the pressure in the body; flowing a filtered inert gas into the internal volume and out the release valve to flush the product with the filtered inert gas and thereby remove contaminants present in the product through the release valve with the filtered inert gas; flowing a sterilizing gas into the internal volume and out the release valve to flush the product with the sterilizing gas, wherein the sterilizing gas passes through a screen before flowing into the internal volume; closing the release valve once a target sterilizing gas concentration has been detected at the release valve; pressurizing the body using the sterilizing gas to a pressure of at least 30 psi for a pressure cycle time to perform a sterilization cycle; selectively venting the release valve during the sterilization cycle to provide a controlled release of an amount sterilizing gas to measure a concentration of the sterilizing gas in the released amount of sterilizing gas; and opening the release valve to fully release the pressure in the body after the sterilization cycle, thereby providing a sterilized product within the internal volume.

### BRIEF DESCRIPTION OF THE DRAWINGS

FIG. 1A is a bottom perspective view of a sterilizer unit in accordance with the disclosure;

FIG. 1B is a top perspective view of the sterilizer unit of FIG. 1A;

FIG. 1C is a top view of the sterilizer unit of FIG. 1A;

FIG. 1D is a front view of the sterilizer unit of FIG. 1A;

FIG. 1E is a rear view of the sterilizer unit of FIG. 1A;

FIG. 1F is a view through section A-A of FIG. 1D;

FIG. 1G is a zoom-in view of circle section D of FIG. 1F.

FIG. 2 is a schematic illustration of a sterilizer unit showing gas flow in accordance with embodiments of the disclosure;

FIG. 3A is a bottom perspective view of a sterilizer unit in accordance with the disclosure;

FIG. 3B is a top perspective view of the sterilizer unit of FIG. 3A;

FIG. 3C is a top view of the sterilizer unit of FIG. 3A;

FIG. 3D is a side view of the sterilizer unit of FIG. 3A;

FIG. 3E is a front view of the sterilizer unit of FIG. 3A;

FIG. 3F is a section view through Section A-A of FIG. 3D;

FIG. 4A is a bottom perspective view of a sterilizer unit in accordance with the disclosure;

FIG. 4B is a top perspective view of the sterilizer unit of FIG. 4A;

FIG. 4C is a top view of the sterilizer unit of FIG. 4A;

FIG. 4D is a side view of the sterilizer unit of FIG. 4A;

FIG. 4E is a front view of the sterilizer unit of FIG. 4A;

FIG. 4F is a section view through Section A-A of FIG. 4D;

FIG. 4G is a zoomed-in view of Section D of FIG. 4F;

FIG. 5A is a top perspective view of a collar for a sterilizer having a screen for flow of the inert gas and sterilizing gas into the sterilizer unit;

FIG. 5B is a bottom perspective view of the collar of FIG. 5A;

FIG. 5C is a top view of the collar of FIG. 5A;

FIG. 5D is a section view through section A-A of the collar of FIG. 5C;

FIG. 5E is a front view of the collar of FIG. 5A;

FIG. 5F is a review view of the collar of FIG. 5A;

FIGS. 6A-6C are screens for a collar for a sterilizer unit in accordance with the disclosure;

FIG. 7A is a top perspective view a vaporizer unit in accordance with of the disclosure;

FIG. 7B is a bottom perspective view of the vaporizer unit of FIG. 7A;

FIG. 7C is a top view of the vaporizer unit of FIG. 7A;

FIG. 7D is a section view of through section A-A of FIG. 7C;

FIG. 7E is a front view of the vaporizer unit of FIG. 7A;

FIG. 7F is a side view of the vaporizer unit of FIG. 7A;

FIG. 8A is a top perspective view of the bottom of the vaporizer unit of FIG. 7A;

FIG. 8B is a bottom perspective view of the bottom of the vaporizer unit of FIG. 7A;

FIG. 8C is a top view of the bottom of the vaporizer unit of FIG. 7A;

FIG. 8D is a section view through section A-A of the FIG. 8C;

FIG. 8E is a zoomed-in view of section B of FIG. 8D;

FIG. 9A is a top perspective view of a heater of the vaporizer unit of FIG. 7;

FIG. 9B is a bottom perspective view of the heater of FIG. 9A;

FIG. 9C is a top view of the heater of FIG. 9A;

FIG. 9D is a bottom view of the heater of FIG. 9A;

FIG. 10A is a top perspective view of an injector of the vaporizer unit of FIG. 7;

FIG. 10B is a bottom perspective view of the injector of FIG. 10A;

FIG. 10C is a top view of the injector of FIG. 10A;

FIG. 10D is a bottom view of the injector of FIG. 10A;

FIG. 11A is a top perspective view of the top of the vaporizer unit of FIG. 7A;

FIG. 11B is a bottom perspective view of the top of the vaporizer unit of FIG. 7A;

FIG. 11C is a top view of the top of the vaporizer unit of FIG. 7A;

FIG. 11D is a front view of the top of the vaporizer unit of FIG. 7A;

FIG. 11E is a section view through section A-A of FIG. 11D; and

FIG. 12 is a schematic illustration of a sterilization unit with a vibratory feeding and bagging system.

### DETAILED DESCRIPTION

It has advantageously been found that the sterilizer unit and methods of sterilizing of the disclosure can safely and effectively sterilize flowable products. In particular, the sterilizer unit and methods of the disclosure can be advantageous for sterilization of cannabis and hemp products. Cannabis and hemp products have interstitial spaces within the buds, which can trap spores, making it difficult to effectively sterilize the products. The unit and methods of the disclosure can advantageously force the sterilizing gas into these interstitial spaces through the use of differential positive pressures established during the sterilization cycle to more effectively sterilize the product. The sterilizer unit and methods of the disclosure can also be gentler on the product, particularly cannabis and hemp products, which can be particularly sensitive to external stimuli, such as the sterilizing gas. It has been advantageously found that the sterilizer unit of the disclosure having the screen interposed between the gas inlet and the internal volume can ensure that the sterilization gas entering the internal volume remains vaporized and does not coalescence into larger droplets, which can adversely affect the product to be sterilized. Cannabis and hemp products, for example, can be discolored by contact with droplets of sterilizing gas, such as hydrogen peroxide. The sterilizer units and methods of the disclosure can also allow for sterilization of the product in loose form, without having to first contain the product in a sterilization pouch.

In embodiments, the sterilizer unit can be useful for sterilizing cannabis pre-roll product. In such embodiments, the pre-roll product can be sealed in a Tyvek package and terminally sterilized using the sterilizer unit of the disclosure. The resulting sterilized product is a sterile pouch that can be used for longer term storage of the pre-roll products. In embodiments, after sterilization the pouch can be further sealed in or have sealed thereto another barrier layer film and modified atmosphere can be created within the additional barrier layer film, which can aid in prolonging the storage time.

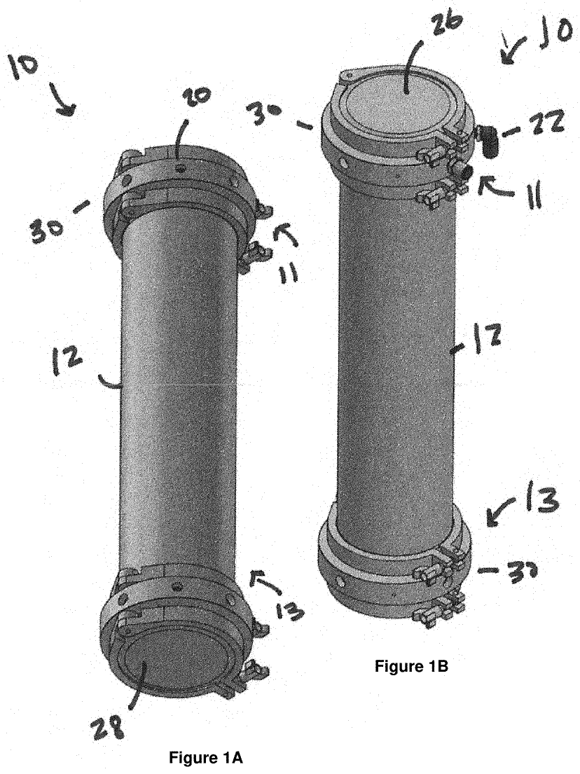

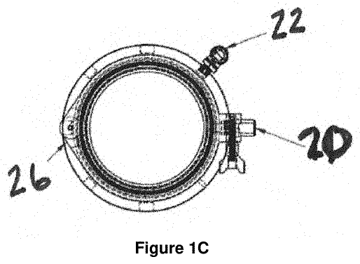

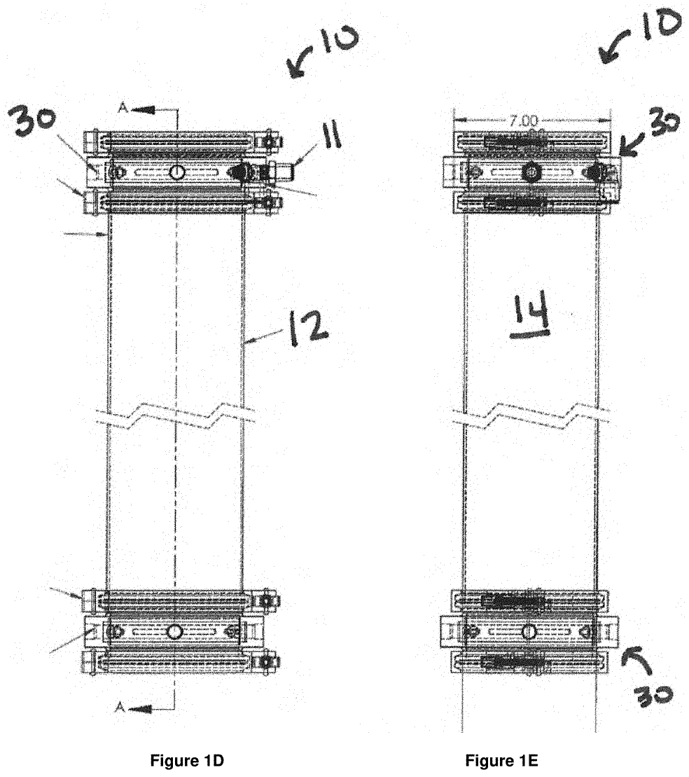

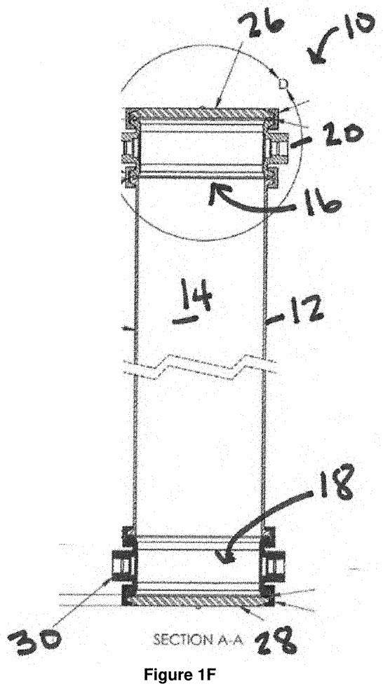

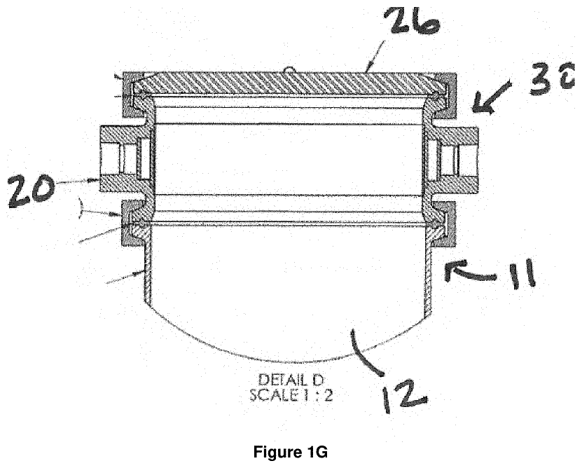

Referring to FIG. 1, in accordance with embodiments, a sterilizer unit 10 in accordance with the disclosure can include a body 12 having an internal volumel4 for receiving the product to be sterilized, a gas inlet 20 in fluid communication with the internal volume for introducing an inert and/or sterilizer gas into the internal volume 14, one or more screens 24 disposed adjacent the gas inlet 20 such that the gas passes through the screen 24 before entering in the internal volume 14, and pressure release valve 22 for releasing the gas from the internal volume 14.

As used herein, the gases that can be flowed through the sterilizer unit 10 include fluids naturally found in gas form, aerosolized reagents, and vaporized reagents. In embodiments, a sterilizer system can include a vaporizer unit 100 in fluid communication with the sterilizer unit 10.

The body 12 of the sterilizer unit 10 can be formed of any suitable material designed to be stable under the target operating pressures. Referring to FIG. 1, the body has a first end 11 and an oppositely disposed second end 13. The product inlet 16 can be disposed at the first end 11 such that the internal volume 14 is filled with the product from the bottom up. In other embodiments, the gas inlet 20 is also located at or adjacent to the first end 11. In such embodiments, the pressure release valve 22 can be disposed at or adjacent to the second end 13.

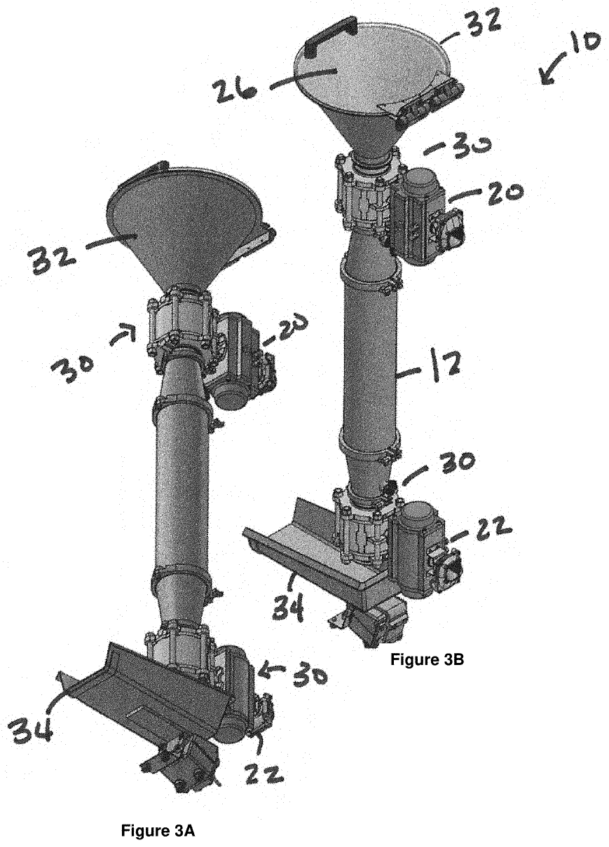

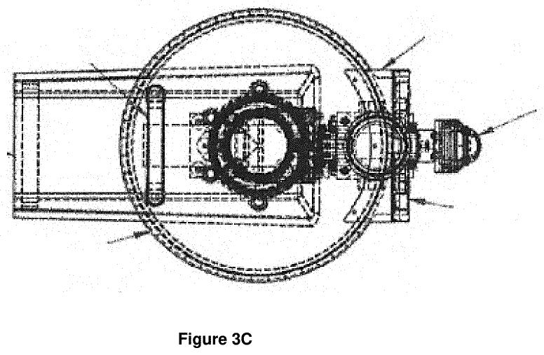

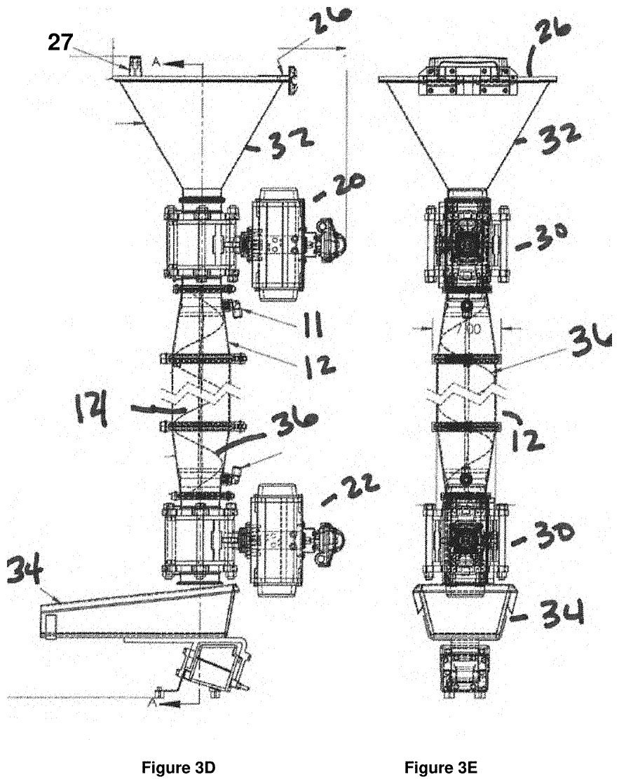

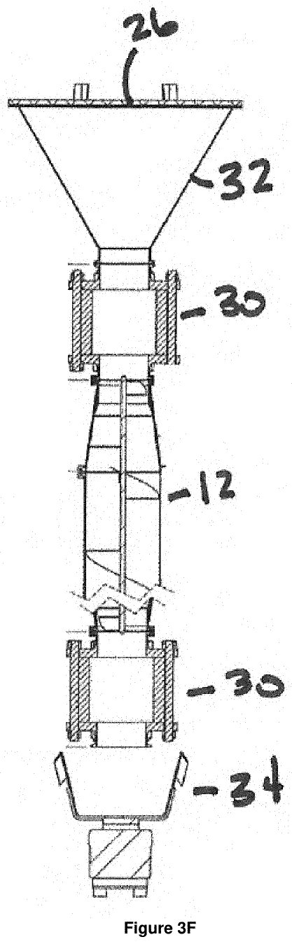

In embodiments, the body 12 is a cylinder as illustrated in FIG. 1. FIG. 3 illustrate an embodiment in which the body has frustoconically shaped tapers at the first and second ends 11, 13. Various shapes and sizes of the body 12 are contemplated herein.

In embodiments, the body 12 can have a length of about 2 inches to about 24 inches, about 4 inches to 20 inches, about 6 inches to about 12 inches, about 2 inches to about 12 inches, about 10 inches to about 24 inches. Other suitable lengths include about 2, 3, 4, 5, 6, 7, 8, 9, 10, 11, 12, 13, 14, 15, 16, 17, 18, 19, 20, 21, 22, 23, 24 inches.

In embodiments, the internal volume 14 defined within the body 12 can have a diameter of about 4 inches to about 36 inches, about 5 inches to about 30 inches, about 10 inches to about 36 inches, about 4 inches to about 12 inches, or about 7 inches to about 25 inches. Other suitable diameters include about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 inches.

The internal volume 14 defined within the body 12 can be sized to accommodate a variety of product volumes to be sterilized. For example, in the cannabis industry, the sterilizer can be sized to accommodate the size of a field pack of the cannabis or hemp product. For example, the internal volume 14 can hold up to about 25 lbs of products. For example, the sterilizer unit 10 can be sized to accommodate about 10 lbs to about 25 lbs of product. Other suitable amounts include about 10, 11, 12, 13, 14, 15, 16, 17, 18, 19, 20, 21, 22, 23, 24, or 25 lbs. Larger and smaller amounts are also contemplated herein. In embodiments, smaller amounts, such as retail packaging quantities on the orders of ounces or grams could also be sterilized herein. This can be advantageous in embodiments for example, where sterilizer unit 10 is provided in-line with a packaging apparatus (not shown).

Referring to FIG. 3, the internal volume 14 can be defined by internal walls of the body 12. In embodiments, the internal walls of the body 12 can have grooves 36 formed therein and/or ribs formed thereon. The ribs and/or grooves 36 can allow for an easy let down of the product during filling of the internal volume with the product. The product can flow along the grooves and/or ribs during filling of the internal volume. In embodiments, the internal volume 14 has cylindrical shape and the grooves 36 and/or ribs spiral down the walls of the internal volume from a top portion near the first end of the body where the product is introduced to a bottom of the internal volume 14 near the second end 13 of the body 12.

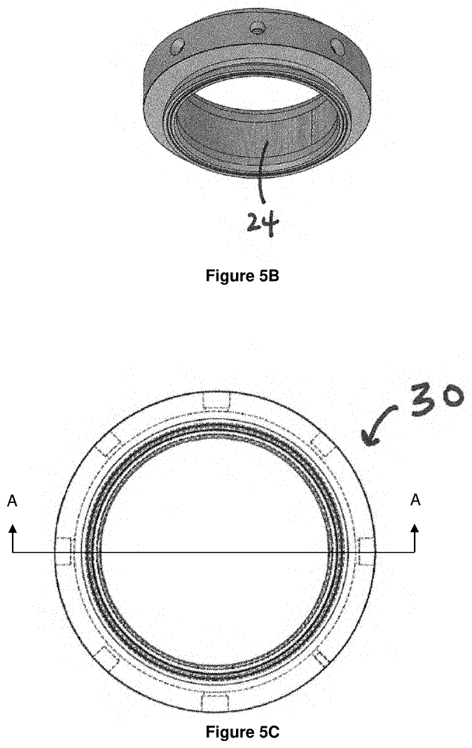

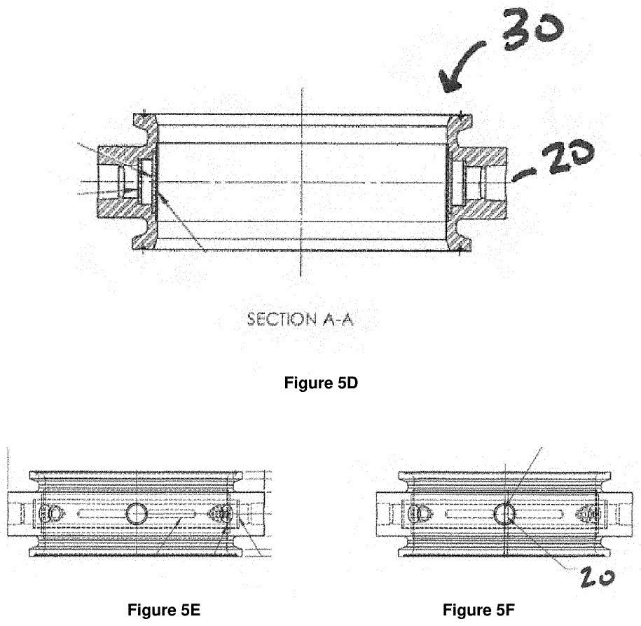

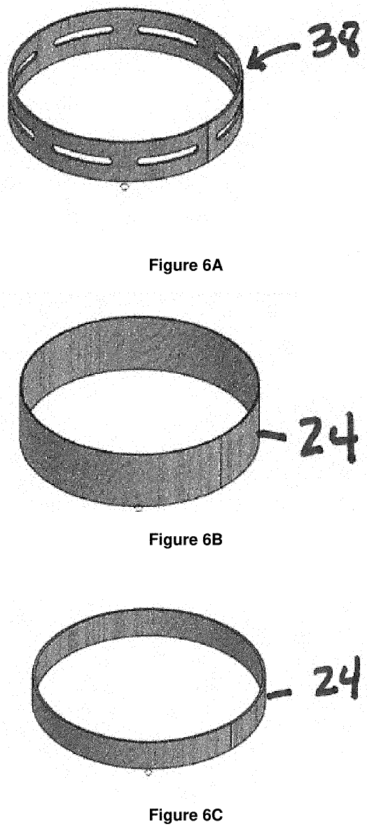

The gas inlet 20 can be provide in fluid communication with the internal volume. For example, as illustrated in FIG. 1, in embodiments, the gas inlet 20 can be provided on a collar 30 disposed on the body 12 at or adjacent to the product inlet 16. In embodiments, gas inlets 20 and/or outlets can be provided on a collar 30 disposed at the product outlet 18 as an alternative to or in addition to the collar 30 at or adjacent to the product inlet 16. For example, a collar 30 at or adjacent to the product outlet 18 can include the pressure release valve 22. The sterilizer unit can include a single gas inlet 20 or multiple inlets. For example, referring to FIGS. 5 and 6, gas inlets 20 can be provided radially spaced around the collar 30. The collar 30 can have an input through which gas from a gas source enters the collar and disperser around to each of the gas inlets 20. In embodiments, the collar 30 can also include openings for receiving one or more sensors, such as concentration sensors, pressure sensors and/or humidity sensors.

The gas inlet 20 can be used for introduction of an inert gas and/or a sterilizing gas. In embodiments, the sterilizing unit 10 includes a plurality of gas inlets each capable of introducing both an inert gas and a sterilizing gas into the internal volume. In other embodiments, the sterilizing unit 10 comprises at least two gas inlets, each of the at least two inlets being for introduction of only one of the inert gas or the sterilizing gas.

In embodiments having a collar 30, the screen can be provided within the collar 30. Referring to FIG. 6, the collar can include a distribution manifold 38 disposed between two screens. The second screen can be disposed inward of the first screen in the direction of flow of the gas into the sterilizer unit. The first screen can have a larger mesh size than the second screen. This arrangement can advantageously provide good radial distribution of the inert gas and/or sterilizing gas, with the second screen restricting the flow to ensure that the particle size of the sterilizing gas vapor remains small. The distribution manifold 38 can in embodiments be formed of a screen or any other suitable material and have a series of slots disposed around the manifold to control the in-flow of gas. Any number of screens can be used in the sterilizing unit.

In embodiments, the one or more screens 24 can have a mesh size of about 0.2 microns to about 1000 micron, about 10 microns to about 50 microns, about 50 microns to about 100 microns, about 500 microns to about 800 microns, or about 700 microns to about 1000 microns. In embodiments, the sterilizer unit can have a first screen that has a 45 micron mesh size and a second screen that has a 70-80 micron mesh size. In embodiments, the one or more screens 24 can be heated. In embodiments, the sterilizer unit 10 can include a heater (not shown) for heating the screen.

In embodiments, the sterilizer unit 10 can include a product inlet door 26 and/or a product outlet door 28 for sealing of the internal volume 14 and allowing access to the internal volume 14 during loading/unloading of the product. The product inlet and outlet doors 26, 28 can be controlled by an actuator to actuate the doors between first and second (open and closed) positions. Either or both of the product inlet door 26 and/or the product outlet door 28 can be provided with a handle 27 for opening/closing the door. An example of a handle 27 is illustrated in FIG. 3D.

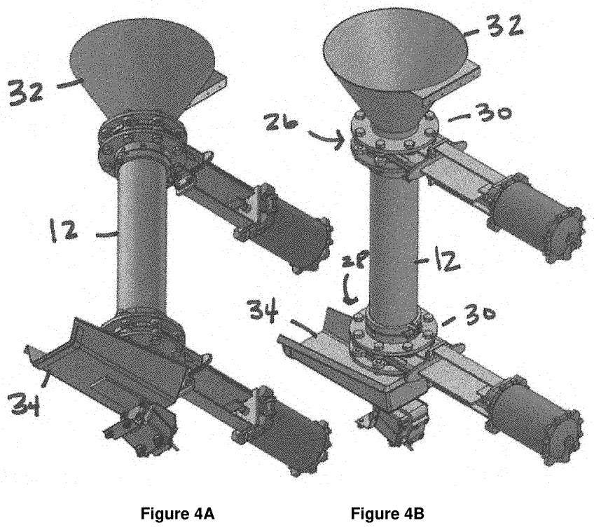

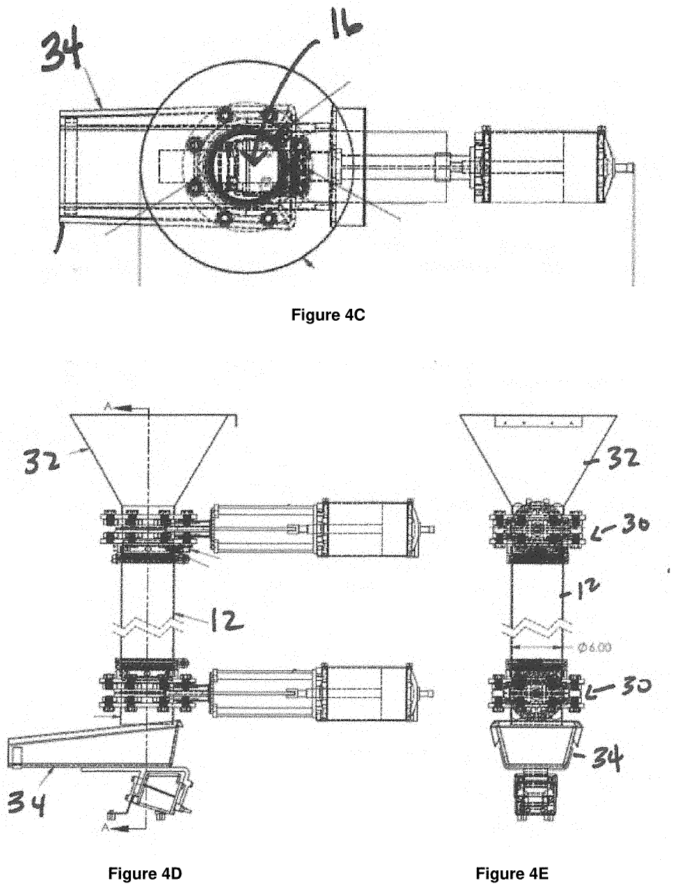

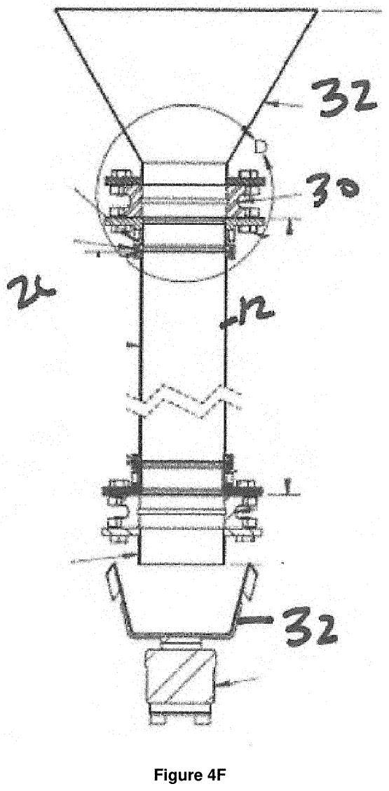

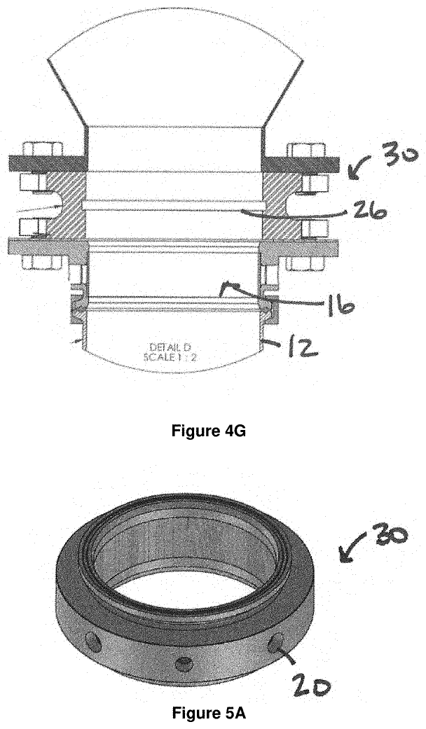

Referring to FIGS. 3 and 4, in embodiments, the sterilizing unit 10 can include a hopper 32 disposed at the product inlet for facilitating loading of the internal volume 14 with the product. The hopper 32 can be sealed by a door that actuates between open and closed positions to expose the product inlet and seal during sterilization. The door sealing the hopper can serve as the product inlet door 26.

In embodiments, a gassing element can be included within the hopper 32 to generate a flow of inert gas across the hopper 32 such that the product passes through the flow of inert gas before entering into the internal volume 14. The hopper 32 further includes an exhaust that captures the flow of inert gas, removing it from the system. The exhaust can be in fluid communication with a filter to filter the removed gas and recirculate filtered gas back to the gassing element. In embodiments, the filter can be a HEPA filter. The gassing element and exhaust can be useful for reducing the contamination load or burden on the product before sterilization. This can include, for example, removing contaminating particulate, as well as removing some of the bacterial, yeast, and/or mold count. The gassing element and exhaust can also be useful in generating an environment within the hopper that has reduced contaminant count, such as bacterial, yeast, and/or mold counts, as compared to the airborne counts in the environment in which the sterilizer is in use.

In embodiments, the door 26 and/or the hopper 32 can further include a UV radiation source for exposing the product to UV radiation prior to entering into the internal volume. This can be useful, like the inert gas for reducing the contamination burden within the product prior to sterilization as well or alternatively creating an improved environment with reduced contamination burden as compared to the airborne counts in the surrounding environment.

In embodiments, the door 26 can include a cutting apparatus. In embodiments, the product to be introduced into the sterilizer unit can be provided in a pouch. The cutting apparatus can be used for opening the pouch to release the product form the pouch and load the product into the sterilizer unit 10.

In embodiments, the sterilizer unit 10 includes a single product inlet and outlet for introduction of the product into the internal volume and release of the sterilized product. In other embodiments, the sterilizing unit includes a product inlet and an oppositely disposed product outlet. The product inlets and outlets 16, 18 can be sealed by the product inlet and product outlet doors 26, 28, respectively. The doors 26, 28can be controlled by an actuator to open and close for entry and release of the product. In embodiments, the product inlet 16 is disposed adjacent the first end 11 of the body 12 and the product outlet 18 is disposed at the second end 13 of the body 12.

In embodiments, the product outlet 18 can be in fluid communication with a packaging machine (not shown). For example, the product outlet can be in fluid communication with a feeder for feeding product to packaging equipment or other element of the packaging equipment such as a hopper, funnel, scale or the like. Referring to FIGS. 3 and 4, in embodiments, a feeder 34 can be arranged at the product outlet, which can be coupled to or otherwise facility flowing the product to another apparatus such as a packaging machine and/or scale. For example, the feeder 34 can allow the product to be flowed into a bulk packaging apparatus. For example, the feeder 34 can allow the product to be flowed to a hopper for weighing and separating into smaller packaging configurations. In embodiments in which the product outlet 18 flows product into an element of the packaging equipment, the sterilizer unit 10 can include a gassing unit that flows filter inert gas over the product as it enters into the packaging equipment to maintain sterilization of the product and maintain low oxygen levels within the product to prevent recontamination. For example, the inert gas can be filtered nitrogen. For example, the inert gas can be HEPA filtered nitrogen.

In any of the sterilization units described herein, the product outlet 18 can include a gate valve for controllable releasing product from the sterilizing unit to a downstream unit.

In alternative embodiments, the product outlet 18 can be configured to release the product directly for field packaging. For example, the product outlet 18 can be positioned to release the product to a slide or funnel that would direct the product into a package. For example, the field package can be a flexible package. Any package configuration, such as blow molded containers, jars, cans, and the like, can be used. In the cannabis industry, for example, the sterilized cannabis product can be field packed and stored for later packaging in smaller retail packaging amounts. In embodiments, the gassing unit can be provided at the product outlet to flow an inert gas over the product outlet as the product is flowed into a package. The gassing unit can include a gassing element to flow the inert gas across the product outlet such that the product passes through the flow as it is released from the sterilizer unit 10 into the package. The gassing unit can further include a gassing element directed to flow inert gas into the package to remove contaminants from the interior of the package and/or provide a modified atmosphere within the package as the product is released from the sterilizer unit into the package. The gassing unit provided to direct gas into the packaging can advantageously provide a modified atmosphere packaged product that resists growth of mold, yeast and/or bacteria while the product remains in the package.

In embodiments, a sealing apparatus can be positioned in close proximity to the sterilizer unit in such embodiments to allow for sealing of the package. The sealing apparatus can include gassing elements to provide a modified atmosphere within the package that maintained within the package during sealing. Any known sealing and/or modified atmosphere machines can be used. The sealing apparatus can be entirely separate from the sterilizer unit. The sterilizer unit, the packaging apparatus, such as the slide or funnel for field packaging, and the sealing apparatus can form a system for field packaging in embodiments.

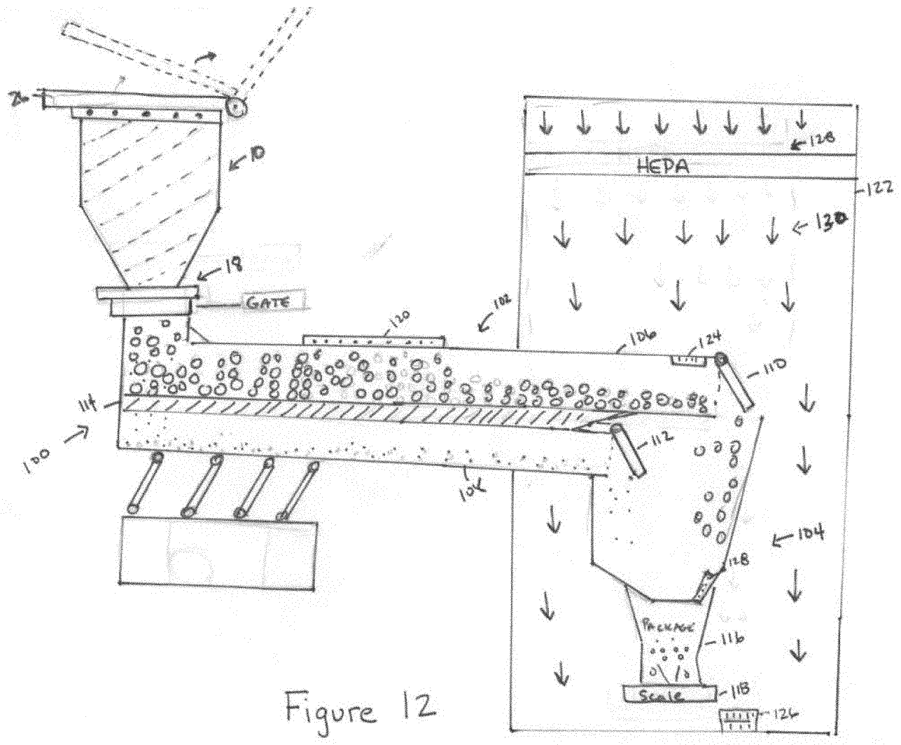

Referring to FIG. 12, the product outlet 18 can be configured to release the product to a vibratory feeding and bagging system 100. In embodiments, the product outlet 18 can include a gate for controllably releasing product from the sterilizer unit 10 into the vibratory feeding and bagging system 100. The vibratory feeding unit 102 can be sealed by a moveable sealing apparatus or door 110,112 that can allow the product to pass from the vibratory feeding unit 102 into the bagging unit 104 when desired, but otherwise seal off the vibratory feeding unit 102. For example, the vibratory feeder unit 102 can have a bulk dribble system 110, 112 that can allows for flow of the product 14 to the bagging system 104 at multiple rates to aid in the efficient and accurate filling of the package to the desired weight. Bulk dripple systems can allow a first rate of product to flow out of the feeder by opening the sealing door 110, 112 to a fully open position until the package weight is close to being met, and then reducing the flow rate of the product by closing the sealing door a portion of the way closed to provide more precise control over the filling process.

In any of the vibratory feeding and bagging systems 100 herein the moveable sealing apparatus 110, 112 can completely seal off the vibratory feeding unit 102 from the bagging unit 194. This can be useful, for example, in isolating the vibratory feeding unit 102, which can be sealed off from the sterilizer unit 10 by the product outlet door, so that the interior of the vibratory feeding unit 102 can be cleaned and/or sterilized when needed by passing filtered inert gas and/or sterilizing gas through the interior of the vibratory feeding unit 102. The interior of the vibratory feeding unit 102 can include one or more vents 124 providing an outlet through which the inert and/or sterilizing gas can be controllably removed and contained from the unit 102. The one or more vents 124 can be in fluid communication with a filter (not shown), such as a HEPA filter, to filter the gas as it exits the interior volume of the vibratory feeder either to recycle the gas into the system for subsequent use or safely remove the gas from the system. The vibratory feeding unit 102 can also include one or more gassing units 120 to maintain a flow of inert gas over the product 14 during the bagging operation. All or a portion of the vibratory feeder and bagging system can be contained in an enclosure 122 and a flow of filtered inert gas 130 can be circulated through the enclosure 122. For example, a portion of the vibratory feeding unit 102 having the moveable sealing apparatus 110, 112 and the bagging system 104 can be contained in the enclosure 122 and have flow of filtered inert gas blown over these systems during operation. The filtered inert gas can be, for example, HEPA filtered inert gas. The enclosure 122 can have a vent 126 at the downstream end of the flow of inert gas to vent the enclosure. The vent can be in communication with a filter, such as a HEPA filter to allow for the vented gas to be filtered and recirculated into the system. The gassing unit 128 of the enclosure 122 can also be adapted to flow sterilizing gas into the enclosure to allow for cleaning and/or sterilization of the enclosure when needed.

The vibratory feeding and bagging system 100 can include, for example, a single vibratory feeder that directs the released sterilized product to a bagging unit 104 that is adapted to receive a desired weight of product and then release it into a package, such as a plastic bag.

As illustrated in FIG. 12, the vibratory feeder and bagging system 100 can alternatively include a vibratory feeding unit 102 that has two or more vibratory feeders 106, 108 that direct the released sterilized product to the bagging unit 104. The vibratory feeder and bagging system 100 can include, for example, two vibratory feeders 106, 108 stacked one on top of the other. The sterilized product can be released from the product outlet 18 to the first vibratory feeder 106. The first vibratory feeder 106 can include a bottom surface that is defined by a screen 114 adapted to allow product of a certain size to pass through the screen 114 and into the second vibratory feeder 108. Each vibratory feeder can be sealed by a movable sealing apparatus 110, 112, such as bulk dribble system that is in fluid communication with the bagging unit 104 to allow product from each vibratory feeding unit 102 to be delivered to the package, if desired, or to control release of the product from the vibratory feeders 106, 108 such that product of only a single size is packaged. Any suitable number of vibratory feeders can be included with screens provided as the bottom surfaces of the top and any intermediate feeders to allow for any number of size separations. This can advantageously provide control over the sizing of the final packaged product allowing more precise mixtures of sizes and/or exclusions of certain sizes.

For example, in the cannabis industry, a dual vibratory feeder system such as shown in FIG. 12 can advantageously allow for size separation of larger size buds from smaller buds and pieces, referred to in the industry as “popcorn and shake.” The dual system can be used, for example to package a mixture of larger size buds and popcorn and shake, with specific control over the ratio of the differently sized buds. The dual system can be used to package larger size buds only. The dual system can be used to obtain precise package weights by allowing the popcorn or shake to top off to achieve finer tuning of the package weights with the smaller and lighter product.

In any of the vibratory feeding and bagging systems 100 herein, the bagging unit 104 can include a scale 126 upon which a package rests and measure the package weight as the package is being filled. Other scale systems can be used, for example, allowing a measured weight of product to be delivered to a scale system from the vibratory feeders before being passed to the package.

The bagging system can further include one or more gassing elements 128 to direct a flow of inert gas around the packaging and/or into the package if modified atmosphere packaging is desired.

The sterilizer unit 10 can be a single unit, integral with the vibratory feeding and bagging system 100 or other downstream packaging system. The sterilizer unit 10 alternatively can be separable from, but attachable to the vibratory feeding and bagging system 100 or other downstream packaging system. This can be make the overall system more mobile and to allow for set-up of the system in various sites, such as at cannabis grower facilities.

Any of the systems herein can be particularly advantageous in the cannabis industry for field packaging cannabis and/or hemp. Field packaging can generally occur in this industry in an environment that can have high contents of airborne contaminants. Field packages can be susceptible to growth of yeast, mold, and/or bacteria within the package as a result of this environment. The sterilizer unit and methods of the disclosure can advantageously provide a sterilized product and a packaging environment that significantly reduces the content of airborne contaminants around the product outlet and within the package during loading of the cannabis and/or hemp from the sterilizer product into the field package. Further, introduction of the inert gas, such as nitrogen into the package for modified atmosphere packaging can be further useful in resisting any growth of yeast, mold, and/or bacteria that may remain within the product after sterilization while the product resides within the package. In any of the foregoing embodiments, the inert gas can be filtered. In embodiments, the inert gas can be HEPA filtered air and/or HEPA filtered nitrogen. In embodiments, the field packages filled using the system of the disclosure can resist yeast, bacteria and/or mold growth for at least sufficient time for determination of a level of contamination if any remains in the product after the sterilization cycle. For example, the filed packages can be resist any change in contamination level for 5 days or more, 7 days or more, 10 days or more from the time of packaging.

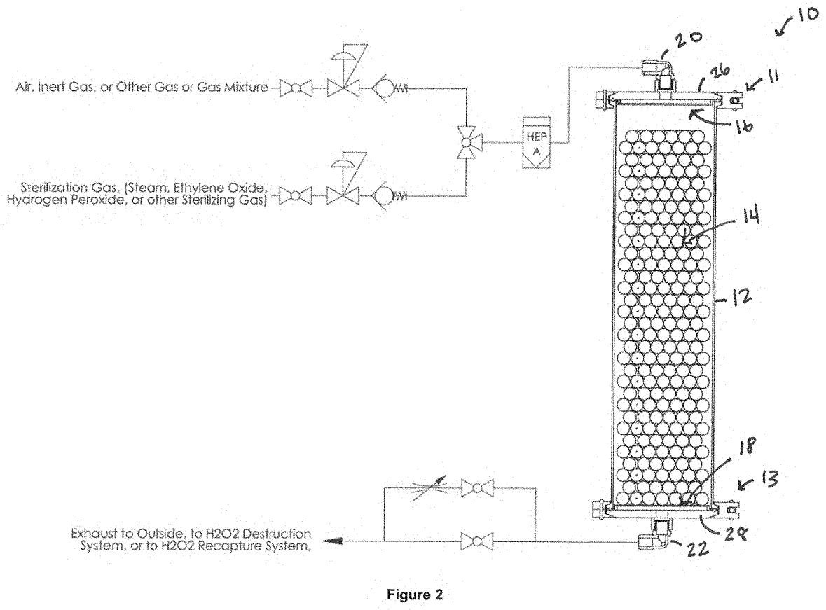

Referring to FIG. 2, in embodiments, the inert gas and sterilizing gas can be flowed into the internal volume 14 from a top portion near or at the first end 11 of the body 12 to a bottom portion of the internal volume 14 near the second end 13 of the body 12. In embodiments, the gas inlets can be disposed at the top portion, while the release valve 22 can be disposed at or adjacent to the bottom portion. In such embodiments, the flow of gas through the entire volume can capture contaminant and force them to the bottom portion, assisted by gravity, and removed through the release valve. This is particularly useful during purge cycles where by the gas is vented through the internal volume and intended to capture contaminants, such as bacteria, yeast, and mold spores, and flow them out of the internal volume through the release valve. It is also contemplated herein, however, that other arrangements of the gas inlet and release valve can be used.

Filtration, such as HEPA filtration can be provided in fluid communication with the release valve 22 to allow for recirculation of the inert gas or sterilization gas in the system. In embodiments, the sterilizer unit can include an inert gas release valve and a sterilizing gas release valve, which can be independently operated depending on which gas is being released from the internal volume. In embodiments, one or both of the inert gas release valve and the sterilizing gas release valve can be in fluid communication with a filter, for example, a HEPA filter, to filter the released gas. The filter can be in fluid communication with the gas inlet to allow for recirculation of the filtered released gas through the unit. In embodiments, the release valve 22 (or sterilizing gas release valve) can be in fluid communication with a filter, for example a HEPA filter, which is then fluid communication with a storage container to store the sterilization gas for future recirculation into the system or disposal. In alternative embodiments, the release valve (or sterilizing gas release valve) can be in fluid communication with storage container with no filter to allow for containment of the released gas for disposal.

In embodiments, a method of sterilizing a product can include introducing the product into the internal volume 14 of the sterilizer body 12 and sealing the internal volume 14 at the product inlet 16 and product outlet 18. The method can include an initial inert gas purge that includes flowing an inert gas, preferably a filtered inert gas, into the internal volume 14 through the gas inlet 20 with the release valve 22 open to allow the inert gas to flow through the internal volume 14 and flush the product therein with the inert gas. This can aid in removing some contaminants, such as bacteria, yeast, and/or mold, from the product by capturing the particular in the flow of inert gas and forcing it out of the internal volume through the release valve 22. The release valve 22 can be in fluid communication with a filter to filter the released gas prior to exhaust or recirculation into the system. The filter for the gas input and/or in fluid communication with the release valve can be a HEPA filter.

In embodiments, the method can further include a test pressurization step in which the release valve 22 is closed and the internal volume is pressurized with inert gas to ensure that the internal volume is sealed without leaks to allow for pressurization and maintenance of the desired pressure. This step of the process can operate as a safety check to ensure that the internal volume can be properly pressurized before using a sterilizing gas pressurization. A pressure sensor within the internal volume or in communication with the internal volume can be used to measure the pressure in real time. The pressure is released and the inert gas is vented through the release valve after the pressurization is confirmed.

The method can then further includes a sterilization gas purge in which sterilization gas is flowed from the product inlet into the internal volume with the release valve open. This allows sterilization gas to interact with the product, build to a target concentration of sterilizing gas within the internal volume 14 before pressurization, and flow out any contaminants that may be captured in the flow to further reduce the contaminant burden present during the sterilization pressurization. The sterilization gas passes through the screen to ensure that the sterilization gas entering the internal volume 14 remains vaporized and does not coalescence into larger droplets, which can adversely affect the product to be sterilized.

A sensor can be arranged at the release valve to measure a concentration of sterilization gas in the outflow from the internal volume during the sterilization gas purge. For example, a collar 30 can include an opening through which a sensor can be disposed and arranged to contact the released gas. The collar 30 can further include a humidity sensor and humidity can be monitored also by measuring the humidity of the outflow from the internal volume and adjusted in real time as needed. The purge can be continued until a target concentration of sterilizing gas is achieved within the internal volume 14 as measured by the sensor at the release valve.

The sensor can be further configured or additional sensors can be included to measure temperature and/or humidity at the release valve. For example, a sensor at the release valve can be configured to measure concentration of sterilizing gas, relative humidity, and temperature. It has advantageously been found that maintaining low humidity levels can improve sterilization efficiency and/or consistency particularly for cannabis and hemp products. The humidity levels within the sterilizer unit 10 can be controlled, for example, using a purge cycle of inert gas with a controlled humidity amount. For example, a purge of 0% R.H. nitrogen can be used to reduce the humidity levels within the sterilizer unit 10.

After the sterilization gas purge, the release valve can be closed to allow the internal volume to be pressurized with the sterilization gas. A small controlled leak of the gas can be provided for by the release valve 22 to allow a sensor outside of the internal volume 14 to measure the concentration of sterilization gas exiting the internal volume 14. This small controlled leak can be allowed to remain throughout the sterilization process or can be opened and closed controllably to check the concentration of sterilization gas within the internal volume 14. The internal volume can be pressurized to at least 15 psi, at least 20 psi, at least 25 psi, or at least 30 psi for a given pressure cycle time to perform the sterilization cycle. Without intending to be bound by theory, increased pressure is believed to result in greater kill percentage of contaminants and/or reduced cycle time. Selection of suitable pressures can be selected based on desired cycle times and kill rates. Once the desired pressure cycle time is achieved, the pressure can be released, venting the sterilization gas through the release valve 22.

In embodiments, after the sterilization cycle, filtered inert gas can be flowed through internal volume 14 and vented through the release valve 22 to flush any remaining sterilization gas out of the internal volume 14. This cleaning or rinsing cycle can include one or more pressure cycles with inert gas as well as purge cycles with inert gas. The pressure cycles can be useful for forcing the inert gas into the interstitial spaces of the product, forcing any sterilizing gas out of the interstitial spaces. This can be advantageous for sensitive products such as cannabis and/or hemp to ensure that sterilizing gas does not remain in contact with the product for longer than necessary and is removed before any subsequent packaging.

The sterile product can then be released from the internal volume 14 through the product outlet 18. In embodiments and for some products, removal of residual sterilization gas may not be important and the final filtered inert gas flush can be omitted.

Any suitable number of purge cycles and pressure sterilization cycles can be included in the methods of the disclosure to achieve the sterilized product. The selection of the number of cycles can be selected depending on the product and the initial contamination burden. The contamination burden can include the presence of mold, fungus, yeast, bacteria, viruses, or other such contaminants.

For example, in an embodiment a method of sterilizing a product can include an inert gas flush during which preferably HEPA filtered nitrogen or air is flowed through the product in the internal volume 14 and vented out an open release valve 22. The process can then include a safety cycle in which the internal volume 14 is sealed, the release valve 22 being closed and pressurized with the inert gas to ensure that the desired pressurization within the internal volume 14 can be achieved. The inert gas can then be released, releasing the pressure. The method can then further include a sterilization gas purge. For example, vaporized H₂O₂ can be flowed into the internal volume 14 through the screen 24 with the release valve 22 open so that the flow of gas is vented out of the internal volume 14. The flow of sterilizing gas can be maintained for a time to achieve a concentration of sterilizing gas of about 100 to 1000 ppm, about 200 ppm to about 600 ppm, or about 400 to about 500 ppm as measured by a sensor arranged to sense the concentration of the released gas. Once the desired concentration is achieved, the internal volume 14 can be sealed again for pressurization with the sterilizing gas to perform the sterilization cycle. A controlled vent of the release valve 22 can be maintained during the sterilization cycle to ensure the desired concentration of the sterilizing gas remains within the internal volume 14. The controlled vent can be opened and closed throughout the sterilization cycle to check the concentration at any desired intervals.

In embodiments, the sterilization cycle can include a first pressure cycle at a first pressure, a second pressure cycle at a second pressure, and a third pressure cycle at a third pressure. For example, the sterilization cycle can include a first pressure cycle at 30 psi, a second pressure cycle at 60 psi, and a third pressure cycle at 20 psi. Any number of pressure cycles and any combination of pressures, which can be the same or different among the pressure cycles can be used. The pressure of the internal volume can be about 20 psi to about 60 psi. Any suitable pressure can be selected within this range or higher so long as the pressure is not higher than the safety limit of the sterilization unit. Generally, the pressure cycles will not be performed at pressures above 150 psi for safety purposes.

During each pressure cycle, the pressure is held for a pressure cycle time. The pressure cycle time can be the same or different for each pressure cycle. The pressure cycle time can be, for example, about 30 seconds to about 60 mins, about 1 min to about 10 mins, about 30 mins to about 60 min, or about 10 mins to about 60 mins. Between each pressure cycle, the pressure is released and sterilizing gas is vented through the release valve 22. In embodiments, the sterilizer unit 10 can have the release valve 22 in fluid communication with a filter to filter the sterilizing gas and recirculate it back into the system for additional pressure cycles.

After the sterilization cycle, the method can include rinsing the internal volume by flowing a filtered inert gas through the product in the internal volume 14. In embodiments, the rinsing can include pressurizing the internal volume 14 one or more times with the filtered inert gas and venting the pressure through the release valve 22. The pressure cycles during the rinsing process can aid in forcing the sterilization gas out of the interstitial spaces of the product to ensure the product is free of sterilizing gas before packaging. This can be particularly important for products such as cannabis or hemp, which can be damaged by prolonged exposure to sterilizing gas. The sensor at the release valve 22 can be used during the rinsing cycle to measure the content of the sterilization gas in the outflow of gas from the internal change. Any number of inert gas flushes and/or pressurization cycles with inert gas can be performed to achieve a desired reduction in the concentration of sterilizing gas as measured by the sensor.

In any of the embodiments herein, the inert gas can be air, nitrogen, argon, carbon dioxides, and mixtures thereof. In embodiments, the inert gas is filtered. In embodiments, the inert gas is HEPA filtered.

In any of the embodiments herein, the sterilizing gas can be vaporized H₂O₂, ethylene oxide gas, or ozone. The sterilizing gas, for example, H₂O₂, can be HEPA filtered for recirculation through the system as discussed above in various embodiments.

In embodiments, the method results in a sterilized product having a contaminant value of less than 10,000 cfu/gram. The contaminant can include any one of yeast, mold, bacteria, virus, and fungas, and particular yeast, mold, and bacteria.

In embodiments, the method results in a field package product which can maintain a contaminant level of less than 10,000 cfu/gram for a storage time of at least 5 days, at least 7 days, at least 10 days, or more.

In any of the embodiments herein, when processing cannabis, hemp, or CBD, the method can further include adding terpenes or flavorings to the internal volume after the sterilization cycle and rinsing cycle has been complete. The terpenes or flavorings can be added in a vaporized or aerosolized form for example with or without the flow of inert gas and the internal volume can be pressurized similarly to the pressure cycles described above for sterilization to allow the terpenes or flavorings, which can result in enhanced penetration of the terpenes and flavorings into the product. Any of the pressures and pressure times disclosed above, as well as any number of pressure cycles can be used for imparting the terpenes or flavorings into the product.

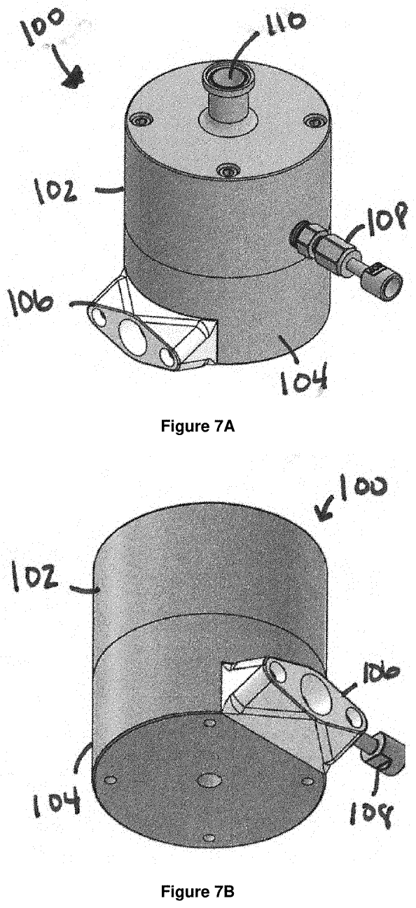

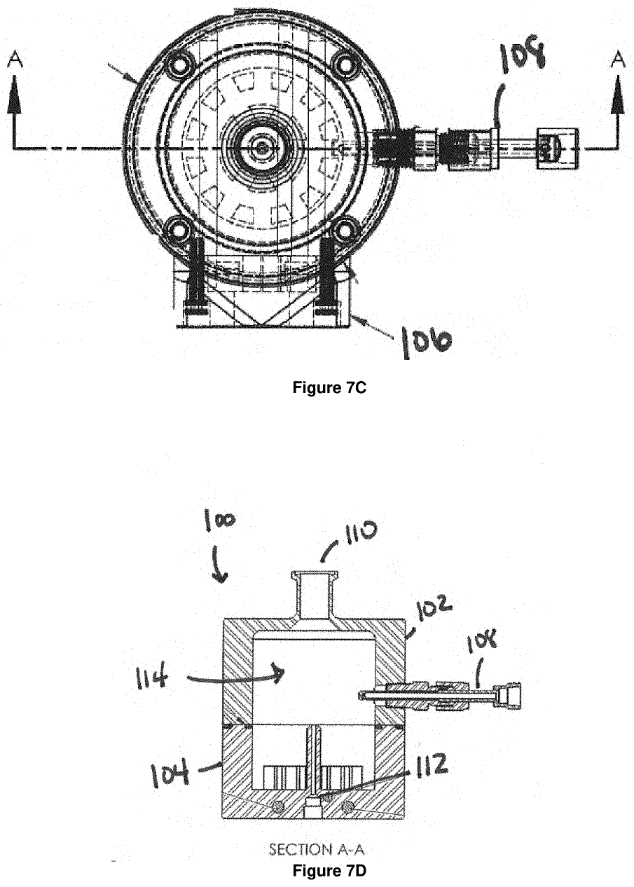

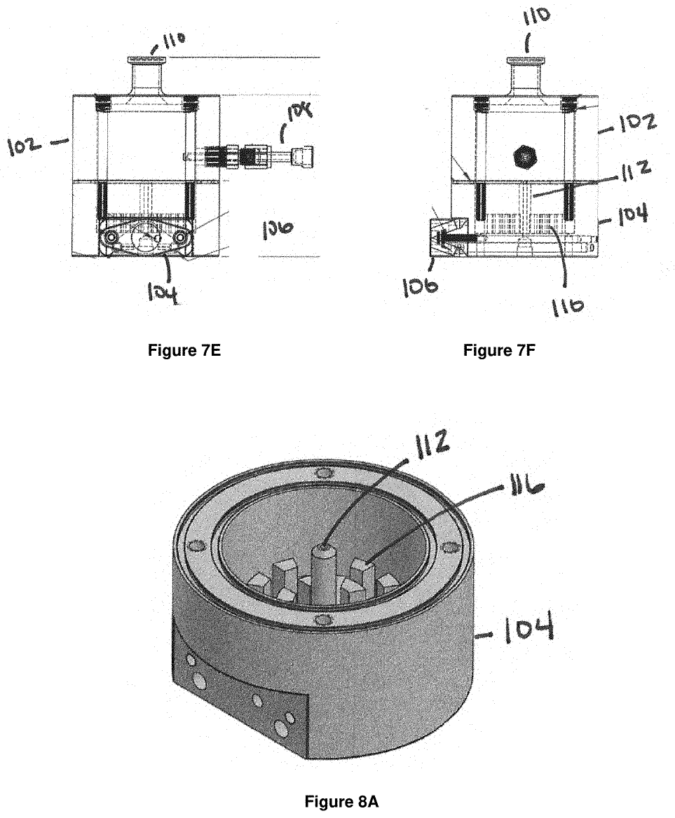

In embodiments, a sterilizer system can include the sterilizer unit 10 of any of the foregoing embodiments and a vaporizer unit 100. Referring to FIG. 7, the vaporize unit 100 can include a heater 106 arranged to heat a bottom 104 region of the vaporizer unit 100, a sterilizing fluid injector 108, and a sterilizing gas outlet 110. The gas outlet 110 of the vaporizer unit 100 can be fluidly coupled to the sterilizer unit 10 gas inlet 20 to deliver the vaporized sterilizing gas under pressure to the internal volume 14 of the sterilizer unit 10.

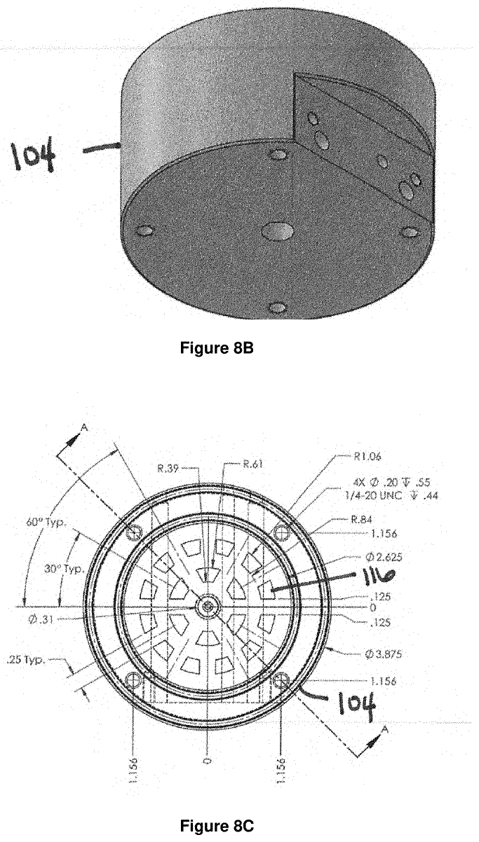

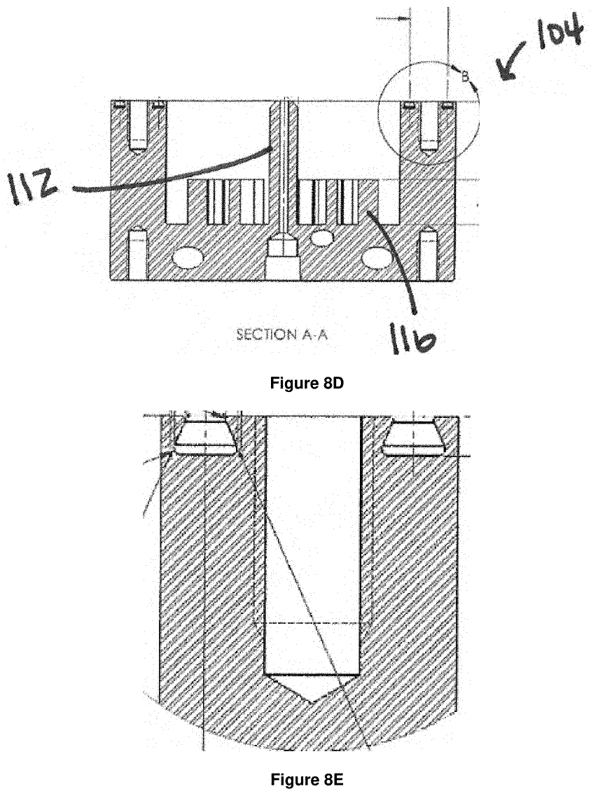

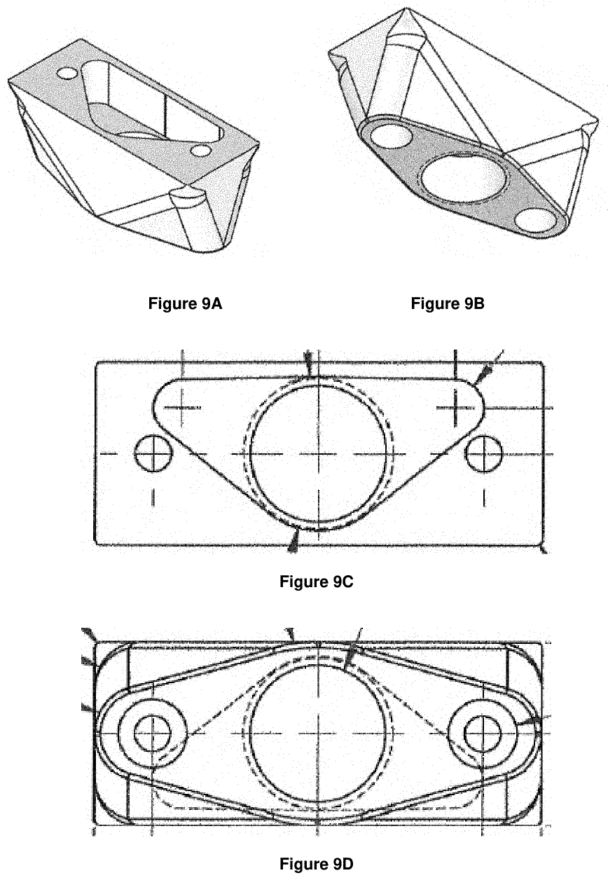

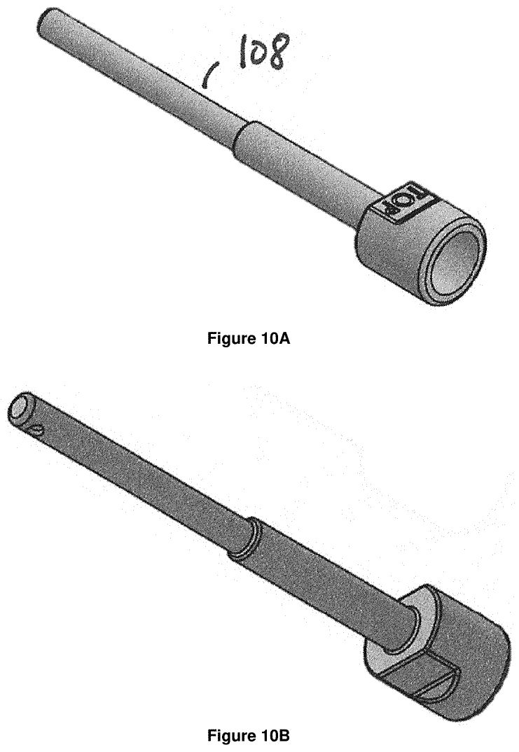

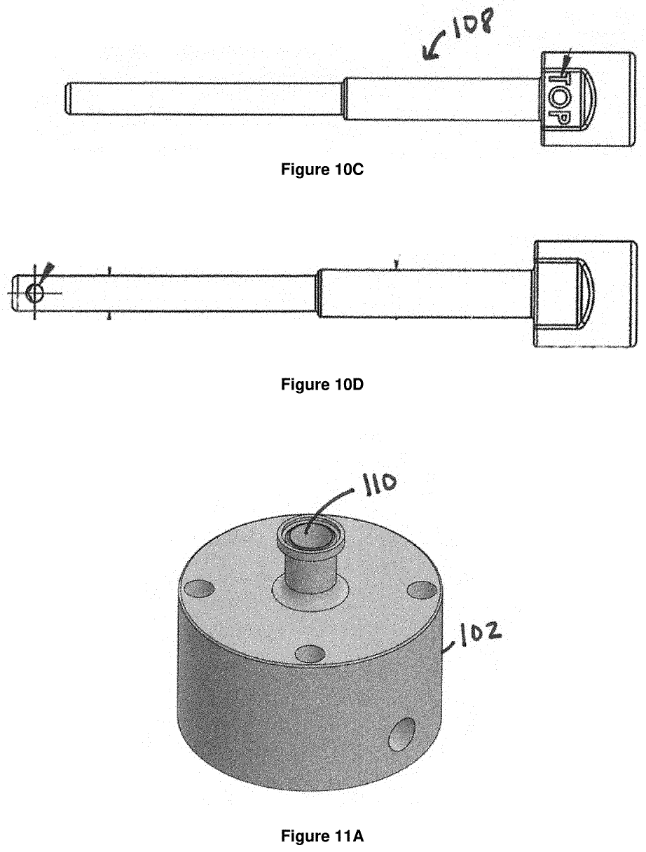

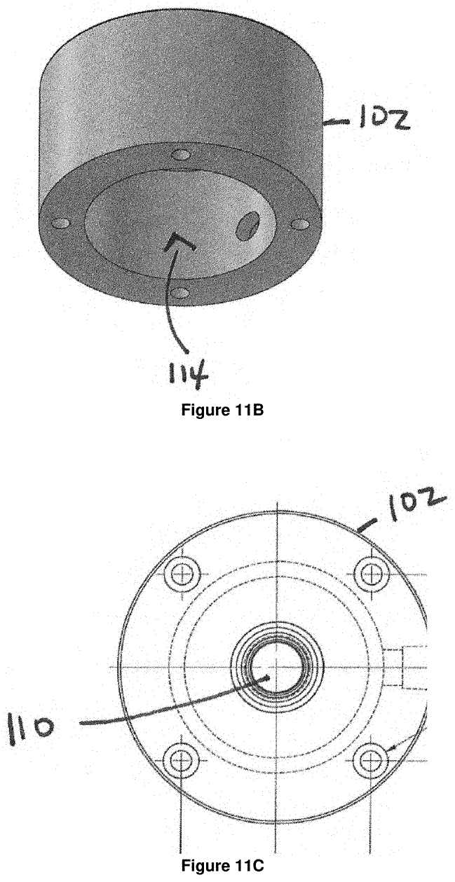

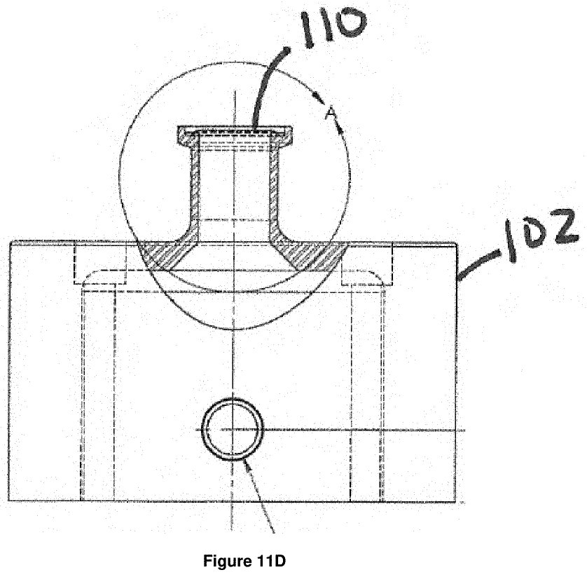

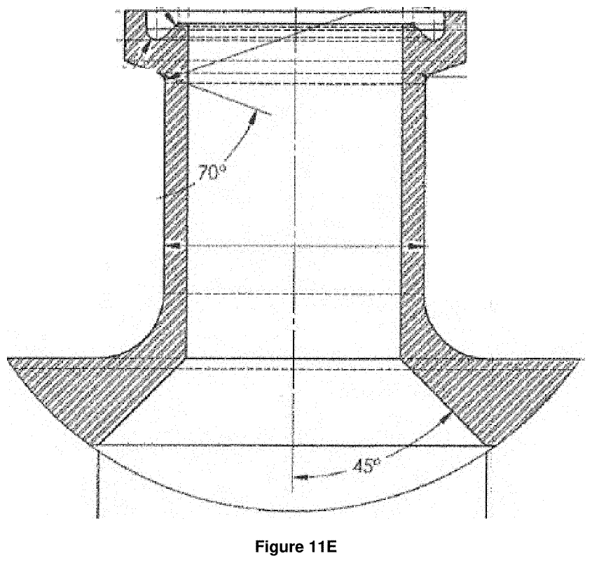

The heater can be a Calrod heater in embodiments with a thermocouple that controls the temperature within the vaporizer unit 100. Referring to FIG. 8, the bottom 104 of the vaporizer unit can be in contact with the heater 106 such that the temperature of the bottom 104 of the vaporizer unit is controllable heated to a temperature using the heater 106. The bottom 104 can include one or more projections extending into the internal volume. The projections 116 can increase the heated surface area that can be contacted by the sterilizing fluid for enhanced vaporization.

The vaporizer unit 100 can further include an inert gas inlet 112 for flowing inert gas into the internal volume of the vaporizer unit 100. Inert gas can be flowed into the internal volume 114 of the vaporizer unit 100 to pressurize or increase the pressure of the vaporized sterilizing gas within the unit 100. Pressurization within the vaporizer unit can be used to control the pressure introduced into the sterilizer unit 10 in embodiments.

In embodiments, the sterilizing gas outlet of the vaporizer unit 100 can be in fluid communication with a nozzle that is fluidly couple with the gas inlet 20 of the sterilizer unit 10 to control the flow of gas into the sterilizer unit 10. In embodiments, the nozzle can also be in fluid communication with an inert gas source, such that a mixture of inert gas and sterilizing gas is provided within the nozzle and delivered under pressure to the sterilizer unit 10 during the one or more sterilization cycles.

In use, the vaporizer unit 100 receives a quantity of sterilizing fluid from the injector 108. The sterilizing fluid can be for example a liquid, for example, liquid H₂O₂ or aqueous solutions of H₂O₂. The sterilizing fluid is then heated through contact with the bottom 104 and projections 116 (if present) of the vaporization unit 100. The bottom 104 is heated to a temperature sufficiently high to vaporize the sterilizing fluid upon contact or residence for a target time period. In embodiments, the bottom 104 can be preheated and small amounts of liquid sterilizing fluid can be introduced into the internal volume 114 such that the liquid almost immediately vaporizes upon contact with any part of the heated bottom 104. In other embodiments, the internal volume 114 can be partially filled with the sterilizing fluid and the bottom 104 can be heated to initiate vaporization once the sterilizing fluid reaches a sufficient temperature for gas or vapor formation. In other embodiments, continuous smaller amounts of fluid can be introduced into the internal volume 114, which reach the temperature for vapor formation more quickly, such that a more continuous cycle of introduction of sterilizing fluid and conversion to vapor is generated.

In any embodiments herein, the preheating of the vaporizer unit 100 can be coordinated with the process performed on the sterilizer unit 10. For example, the preheating and/or boiling of the sterilizing fluid can be performed in the vaporizer unit 100 while the sterilizer unit 10 is undergoing processes involving the inert gas. This can ensure a quantity of sterilizing gas is vaporized within the vaporization unit 100 and ready for introduction into the sterilizer unit 10 when the sterilizer unit 10 has completed the inert gas cycles and is ready for sterilization cycles.

In embodiments, the release valve 22 of the sterilizer unit 10 can be in fluid communication with a filter for recirculation of the outflowed sterilizing gas for subsequent use. In some embodiments, the outflowed sterilizing gas can be condensed to a liquid, filtered, and the recirculated to the vaporizer unit 100 for reuse.

In embodiments, the sterilizing fluid can be liquid H₂O₂. In embodiments, the liquid H₂O₂ can be provided as an aqueous solution, for example, a 30-40% solution of H₂O₂ in water.

The sterilizer unit, sterilizer system, and methods of the disclosure can allow for faster cycle times as compared to conventional vacuum sterilization methods, particularly for cannabis, hemp, and CBD products, can have higher kill rates at short cycle times, can be gentler on the product avoiding degradation of sensitive products, such as cannabis, hemp, and CBD, can have improved removal of the sterilizing gas from the product after sterilization and reduce overall processing time by eliminating the need to vent the product after sterilization.

The use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict embodiments for purposes of illustration only. One of ordinary skill in the art will readily recognize from the following discussion that alternative embodiments of the structures and methods illustrated herein may be employed without departing from the principles described herein.

Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1. A sterilization unit for sterilizing a product, comprising:
a body having an internal volume for receiving the product to be sterilized, and comprising at least one opening through which product can be introduced and/or withdrawn from the internal volume; a gas inlet in fluid communication with the internal volume for introducing one or more of an inert gas and a sterilizing gas into the internal volume during sterilization, wherein the at least one opening is adapted to be sealed for pressurization of the internal volume;
one or more screens disposed in the gas inlet through which the one or more of the inert gas and the sterilizing gas must pass before entering into the internal volume; and
a pressure release valve,
wherein the body is capable of being pressurized to a pressure of 10 atm or more.

2. A sterilization unit for sterilizing a product, comprising:
a body having an internal volume for receiving the product to be sterilized and comprising a product inlet end having a product inlet through which product can be introduced into the internal volume and an oppositely disposed product outlet end having a product outlet through which product can be removed from the internal volume through gravitational flow;
a product inlet door configured to shift between a first position in which the product inlet door seals the body at the product inlet end and a second position in which the product inlet door is disposed away from the product inlet end and exposes the product inlet to allow product to be introduced into the internal volume;
a product outlet door configured shift between a first position in which the product inlet door seals the body at the product outlet end and a second position in which the product outlet door is disposed away from the product outlet end to expose the product outlet and allow product to flow out from the internal volume;
a gas inlet in fluid communication with the internal volume for introducing one or more of air and a sterilizing gas into the body during sterilization;
one or more screens disposed in the gas inlet through which the one or more of the air and sterilizing gas much pass before entering into the body; and
a pressure release valve,
wherein the body is capable of being pressurized to a pressure of at least 10 atm.

3. The sterilization unit of claim 2, further comprising a hopper at the product inlet end of the body, the hopper having a first end and an oppositely disposed second end, wherein product is introduced through the hopper at the first end and flows through the second end and the product inlet into the internal volume, and the product inlet door seals the hopper at the first end.

4. (canceled)

5. The sterilization unit of claim 2 or 3, further comprising a feeder disposed at the product outlet end, wherein when the product outlet door is in the second position, the product flows from the internal volume onto the feeder.

6. (canceled)

7. (canceled)

8. The sterilization unit of claim 1, further comprising a sensor disposed to be adapted to measure a concentration of sterilizing gas from the release valve.

9. The sterilization unit of claim 1, further comprising a heater disposed at or adjacent to the gas inlet to heat the screen and/or the one or more of the inert gas and the sterilizing gas before it enters into the internal volume.

10. (canceled)

11. The sterilizer unit of claim 1, comprising a plurality of gas inlets radially spaced around the body, wherein the screen is disposed radially around the body, covering the gas inlets.

12. (canceled)

13. (canceled)

14. (canceled)

15. (canceled)

16. (canceled)

17. (canceled)

18. The sterilizer unit of claim 16, wherein the product is cannabis or help and the unit is adapted to sterilize about 10 lbs to about 25 lbs of cannabis or hemp in a single batch.

19. A method of sterilizing a cannabis or hemp product, comprising:
introducing the product into an internal volume of a body of a sterilizer unit and sealing the internal volume, wherein the sterilizer unit further comprises at least one gas inlet in fluid communication with the internal volume for pressurizing the body and at least one release valve for releasing the pressure in the body;
flowing a inert gas into the internal volume and out the release valve to flush the product with the filtered inert gas and thereby remove spores present in the product through the release valve with the inert gas;
flowing a sterilizing gas into the internal volume and out the release valve to flush the product with the sterilizing gas, wherein the sterilizing gas passes through a screen before flowing into the internal volume;
closing the release valve once a target sterilizing gas concentration has been detected at the release valve;
pressurizing the body using the sterilizing gas to a pressure of at least 30 psi for a pressure cycle time to perform a sterilization cycle,
selectively venting the release valve during the sterilization cycle to provide a controlled release of an amount sterilizing gas to measure a concentration of the sterilizing gas in the released amount of sterilizing gas; and
opening the release valve to fully release the pressure in the body after the sterilization cycle, thereby providing a sterilized product within the internal volume.

20. (canceled)

21. (canceled)

22. The method of claim 19, wherein the sterilizing gas is vaporized H₂O₂, ethylene oxide, and/or ozone.

23. (canceled)

24. (canceled)

25. The method of claim 19, wherein the product is cannabis or hemp.

26. (canceled)

27. The method of any claim 19, further comprising closing the release valve and pressurizing the body with the inert gas before the sterilization cycle to perform a safety cycle and releasing the pressurized inert gas before the sterilization cycle.

28. The method of claim 19, wherein the sterilization cycle comprises a first pressurizing of the body using the sterilizing gas to a first pressure of at least 15 psi for a first pressure cycle time, releasing the pressurized sterilizing gas after the first pressure cycle time, and a second pressurizing of the body using the sterilizing gas to a second pressure for a second pressure cycle time and releasing the pressured sterilizing gas after the second pressure cycle time.

29. (canceled)

30. (canceled)

31. The method of claim 28, wherein the sterilization cycle comprises a third pressuring of the body using the sterilizing gas to a third pressure for a third pressure cycle time, wherein the third pressure is less than the first and second pressures.

32. (canceled)

33. The method of claim 19, further comprising releasing the sterilized product from the internal volume through a product outlet of the sterilizer unit to a feeder disposed adjacent to the product outlet, wherein when the sterilized product is present in the feeder, a flow of HEPA filtered inert gas is passed over the sterilized product.

34. (canceled)

35. The method of claim 19, further comprising loading the internal volume with the product through a hopper disposed at a product inlet end of the sterilizer unit, while flowing HEPA filtered inert gas over the product in the hopper during loading of the internal volume of the sterilizer unit.

36. (canceled)

37. (canceled)

38. The method of claim 19, wherein the sterilized product has a contaminant level of less than 10,000 cfu/gram, wherein the contaminant is one or more of bacterial, yeast, fungus, virus, and mold.

39. A vibratory feeding and bagging system, comprising:
a vibratory feeding unit comprising:
a first vibratory feeder stacked on top of a second vibratory feeding,
a screen interposed between the first and second vibratory feeders such that product having a size smaller than a mesh size of the screen can pass from the first vibratory feeder into the second vibratory feeder,
first and second bulk dribble systems disposed at an outlet end of the first and second vibratory feeders, respectively, and
one or more gassing units arranged to direct a flow of inert gas over the product in the first and second vibratory feeders; and
a bagging unit in fluid communication with the vibratory feeding unit and adapted to receive product from the first and/or second vibratory feeder for filling a package with the product, the bagging unit comprising one or more gassing unit arranged to direct a flow of inert gas over the package and product being packaged.

40. The vibratory feeding and bagging system of claim 39, further comprising an enclosure enclosing at least a portion of the vibratory feeding unit and the bagging unit, the enclosure comprising one or more gassing units and one or more vents to circulate inert filtered gas through the enclosure.

41. (canceled)

42. A sterilization and bagging system, comprising:
a sterilizing unit in fluid communication with a vibratory and bagging system, the sterilizing unit comprising:
a body having an internal volume for receiving the product to be sterilized, and comprising at least one opening through which product can be introduced and/or withdrawn from the internal volume; a gas inlet in fluid communication with the internal volume for introducing one or more of an inert gas and a sterilizing gas into the internal volume during sterilization, wherein the at least one opening is adapted to be sealed for pressurization of the internal volume,
one or more screens disposed in the gas inlet through which the one or more of the inert gas and the sterilizing gas must pass before entering into the internal volume, and
a pressure release valve,
wherein the body is capable of being pressurized to a pressure of 10 atm or more
the vibratory feeding and bagging system, comprising:
a vibratory feeding unit comprising:
a first vibratory feeder stacked on top of a second vibratory feeding,
a screen interposed between the first and second vibratory feeders such that product having a size smaller than a mesh size of the screen can pass from the first vibratory feeder into the second vibratory feeder,
first and second bulk dribble systems disposed at an outlet end of the first and second vibratory feeders, respectively, and
one or more gassing units arranged to direct a flow of inert gas over the product in the first and second vibratory feeders; and
a bagging unit in fluid communication with the vibratory feeding unit and adapted to receive product from the first and/or second vibratory feeder for filling a package with the product, the bagging unit comprising one or more gassing unit arranged to direct a flow of inert gas over the package and product being packaged,
wherein the vibratory feeding unit is positioned to directly receive sterilized product from the sterilizer unit.
